(19) United States
(12) Patent Application Publication
(10) Pub. No.: US 2026/0162785 A1
(43) Pub. Date: Jun. 11, 2026

(54) METHODS AND SYSTEMS FOR CLAIM EVALUATION

(71) Applicant: Cigna Intellectual Property, Inc., Wilmington, DE (US)

(72) Inventors: Julie B. Kessel, St. Petersburg, FL (US); Parmis Green, Moorestown, NJ (US); Denise Burns, Bloomfield, CT (US); Jennifer Ruiz, Visalia, CA (US); Dayra I. Olson, Bloomfield, CT (US); Tracy Johnson, Bloomfield, CT (US); Paul C. Rossi, Bloomfield, CT (US)

(21) Appl. No.: 19/410,701

(22) Filed: Dec. 5, 2025

Related U.S. Application Data
(60) Provisional application No. 63/728,945, filed on Dec. 6, 2024.
(60) Provisional application No. 63/914,921, filed on Nov. 10, 2025.

(51) Int. Cl. G16H 10/60

(52) U.S. Cl. CPC G16H 10/60

(57) ABSTRACT

Methods and systems for evaluating claims are described. A system includes a large language model (LLM) subsystem configured to (a) adapt code or instructions to learn from a training dataset, (b) receive data comprising procedure data, (c) ingest the procedure data and a self-assigned level of care, and (d) alter feature vectors in response to new data, a prediction subsystem configured to (a) determine a respective score for each procedure, (b) apply a respective weight to each respective score, (c) determine a final score based on the respective weight applied to each respective score, (d) compare the final score to one or more thresholds to determine a predicted level, and (e) compare the predicted level to the self-assigned level; and a level adjuster subsystem configured to amend the data with the predicted level when the predicted level is different than the self-assigned level.

### CROSS-REFERENCE TO RELATED APPLICATIONS

This application claims the benefit and priority of U.S. Provisional Application No. 63/728,945, filed on Dec. 6, 2024, and U.S. Application No. 63/914,921, filed on Nov. 10, 2025. The entire disclosures of each of the above applications are incorporated herein by reference

### FIELD

The present disclosure relates generally to the technical field of data integrity and artificial intelligence/machine learning. In a specific example, the present disclosure may relate to using an artificial intelligence and/or machine learning model to make decisions regarding an evaluation for, and possible correction of data records.

### BACKGROUND

Processing data related to certain medical claims, such as non-admitted emergency room visits, may involve multiple procedures, each of which having a respective procedure code. The codes may be numerous. Depending on the number of procedures performed in an emergency room visit, and depending on the type of patient treated, claims may be fulfilled differently by a company.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a block diagram of an example benefit manager device that may be deployed within the system of FIG. 1, according to an example embodiment;

FIG. 5 is an example medical claim, according to an example embodiment;

FIG. 6 is a block diagram of a flowchart illustrating methods for evaluating a level of care represented by a claim, according to an example embodiment;

FIGS. 7A and 7B are flowcharts depicting an example method for adjusting a code level of a claim, according to an example embodiment;

FIG. 8 is a block diagram of an example patient management platform that may be deployed within the system of FIG. 1, according to some examples; and

FIG. 9 is a functional block diagram of an example neural network that can be used for the model inference engine or other functions (e.g., engines) as described herein to produce various predictive models.

In the drawings, reference numbers may be reused to identify similar and/or identical elements.

### DETAILED DESCRIPTION

### Introduction

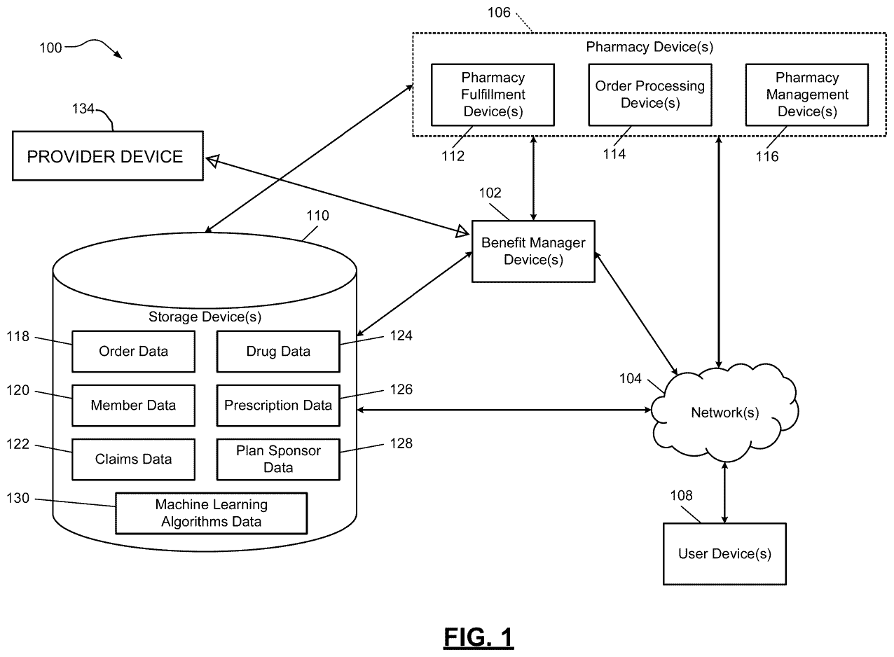

FIG. 1 is a block diagram of an example implementation of a system 100 for a high-volume pharmacy, which is a data processor. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104. The system 100 may also include a storage device 110.

The benefit manager device 102 is a device operated by an entity that is at least partially responsible for creation and/or management of the pharmacy or drug benefit or a medical benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. The benefit manager device 102 can process prescription claims, medical claims (e.g., emergency room medical visits), or both. The benefit manager device 102 processes thousands or tens of thousands of records related to medical claims per day. The processing of this data must be through machine systems as it cannot be processed at a speed or with accuracy as required by humans. In some examples, each claim must be processed in less than 10 seconds, less than 5 seconds, or less than 3 seconds, with a volume that can exceed ten thousand claim records per hour. This volume in view of the rules and requirements not be the same for all records is not a process that can be done accurately by people while meeting the processing speed requirements. The data related to a claim can be received from a provider device 134. The medical claims can be received from the provider device 134, either directly or through the network 104. An issue addressed by the present methods and systems may be that the data record for the claim is reviewed and a decision is made whether to accept the data in the record or if the data should be amended. As discussed herein the medical record can be evaluated by the benefit manager device 102 to adjust the level based at least on the diagnosis data in the record. The adjustment of the claim level is to correct the claim record for further adjudication of the claim record. In another example, the utilization contacted in the medical record can also be used. The system may also apply a weighting to the level stored in the medical record.

In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long-term care benefit, a nursing home benefit, etc. In an example, the medical record may have a drug or pharmacy component. The PBM may, in addition to its PBM operations, operate one or more pharmacies. The pharmacies may be retail pharmacies, mail order pharmacies, etc.

In some embodiments, the benefit manager device 102 can include one or more servers, supercomputers, or other computing device(s) configured to implement artificial intelligence models, such as a feed-forward neural network, a convolutional neural network, a recurrent neural network, a Random Forest model, a Naïve Bayes model, a decision trees model, a logistic regression model, generative artificial intelligence large language model (“LLM”), or any other deep learning and/or machine learning artificial intelligence model. In some embodiments, the benefit manager device 102 can include a group of computers, such as a neural network configured to implement artificial intelligence deep learning and/or machine learning models. The weighting applied to the records can be refined using the machine learning types listed herein. In an example, the adjustment, e.g., upcoding or downcoding the level for an individual record, can be determined using the models and LLMs discussed herein for an individual record.

The benefit manager device 102 can implement evaluation and management service on the records it receives. The device 102 can apply rules and adjust the level of a medical record based on rules, models, or LLM, which can be different for different categories and subcategories of service. In an example, the device 102 evaluates records of face-to-face encounters with the patient and a provider. The categories can be broken down into the following categories: Office or Other Outpatient Services; Telemedicine Services; Hospital Inpatient and Observation Care Services; Consultations; Emergency Department Services; Nursing Facility Services; Home or Residence Services; and Prolonged Service With or Without Direct Patient Contact on the Date of an Evaluation and Management Service. Each of these categories can be an input into the engine (e.g., model, LLM or rules system). A subcategory can be for a new patient or an established patient. The subcategories can also be inputs into the computing engine. The medical record can include a level of services that represents the medically appropriate history, physical examination, or both. The level of service can also be an input into the computing engine. The nature and extent of the history and/or physical examination are determined by the treating physician or other qualified health care professional reporting the medical service. The care team may collect information, and the patient or caregiver may supply information directly (e.g., by an electronic health record (EHR) portal to the database 110 questionnaire). In some embodiment, the gathered information is stored in an electronic record that can be processed by a computing engine. The extent of history and physical examination may not be an element in selection of the level of the evaluation and management service codes that are stored in the record. The evaluation and management section is divided into broad categories, such as office visits, hospital inpatient or observation care visits, and consultations. Most of the categories are further divided into two or more subcategories of evaluation and management services. For example, there are two subcategories of office visits (new patient and established patient) and there are two subcategories of hospital inpatient and observation care visits (initial and subsequent). The subcategories of evaluation and management services are further classified into levels of evaluation and management services that are identified by specific codes, which are stored in the machine-readable medical record.

A format of codes with levels of evaluation and management services based on medical decision making (MDM) or time is the same. First, a unique code number is listed. Second, the place and/or type of service is specified (e.g., office or other outpatient visit). Third, the content of the service is defined. Fourth, time is specified. The place of service and service type are defined by the location where the face-to-face encounter with the patient and/or family/caregiver occurs. For example, service provided to a nursing facility resident brought to the office is reported with an office or other outpatient code. These are all data types in the machine-readable medical record.

The benefit manager device 102 in the case where there is a single diagnosis code can evaluate the medical record as follows. If the ICD-10 diagnosis field in the medical record is reported in medical records for a patient greater than 5 times over a period of a year, 18 months or two years, this can be a chronic condition. The benefit manager device can down code the level if the system assigns a level that is less than the level in the medical record. The down coding can be an automated process employing the computing engine, e.g., electronic circuits implementing tasks for rules, machine models, LLMs, or combinations thereof

Some of the operations of the PBM that operates the benefit manager device 102 may include the following activities and processes. A member (or a person on behalf of the member) of a pharmacy benefit plan may obtain a prescription from or interact with a physician, and the member may seek to receive a prescription drug from a pharmacy or visit a provider for medical treatment. The drug can be part of the medical record. The prescription drug may require prior authorization from a health plan provider prior to dispensing the drug, and the PBM may request prior authorization. Upon receiving authorization approval, the member may obtain the prescription drug. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device 108, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $ 10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in the storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member, receiving authorization approval, and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, plan sponsor data 128, and/or deep learning/machine learning model data 130. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

Furthermore, the deep learning/machine learning algorithms data 130 can include code or instructions necessary to implement each of multiple neural network models and/or machine learning models and/or large language models. The code or instructions can be implemented by one or more processors of the benefit manager device 102 or the pharmacy device 106. Each of the multiple neural network models and/or machine learning models can evaluate claims based on one or more factors (e.g. patient age, patient gender, prescribed drug, patient medical conditions), a process that can be performed each time a claim is submitted. Each of the multiple neural network models and/or machine learning models can include code or instructions to learn from a training dataset, as would be understood by one having ordinary skill in the art. Each of the multiple algorithms can change or adapt the code or instructions based on learning from the training dataset and make predictions according to the changed code or instructions. According to an exemplary embodiment, the multiple machine learning models can include the Random Forest machine learning algorithm, the K-Neighbor machine learning algorithm, the Gaussian Naïve Bayes machine learning algorithm, a decision tree algorithm, a logistical regression model, a generative artificial intelligence LLM model, and the SGD machine learning algorithm. According to an exemplary embodiment, the multiple neural network models can include a feed-forward neural network, a convolutional neural network, and a recurrent neural network.

In some embodiments, each of the multiple neural network models and/or machine learning algorithms can evaluate a claim submitted by, for example, a hospital or other medical facility via the provider device 134. The claim evaluation performed by the multiple neural network models and/or machine learning algorithms can find errors in a medical claim, make changes to the medical claim, add information or columns to the claim (like certain medical coding numbers), or reclassify a medical claim, among other options. By evaluating received claims, each of the multiple neural network models and/or machine learning algorithms can automatically authorize prescription or medical procedure claims, thereby saving time for physicians, pharmacies, and patients in filling a prescription.

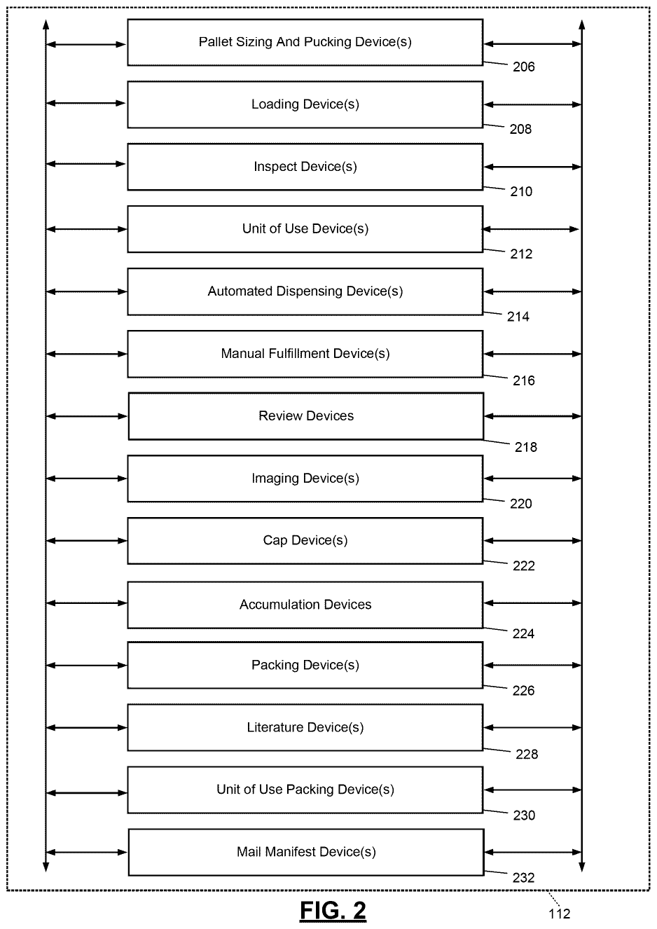

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices. The devices in the system 200 can be controlled based on the level set in the medical records that have been adjusted using the methods and systems herein.

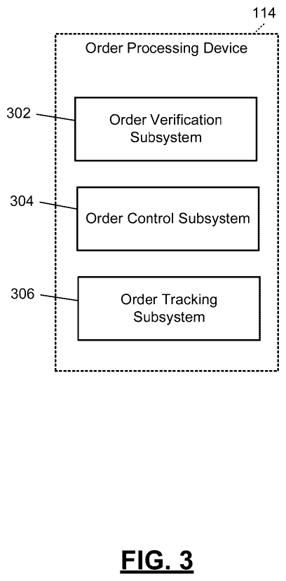

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may include order related components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

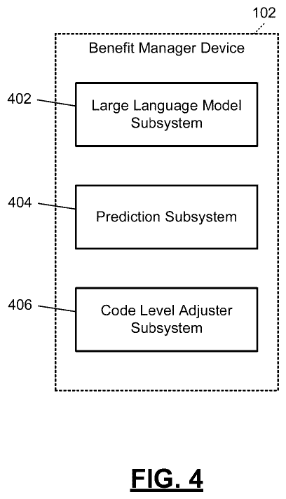

FIG. 4 illustrates the benefit manager device 102, according to an example embodiment. The benefit manager device 102 may be deployed in the system 100, or may otherwise be used. The benefit manager device 102 may include a large language model (“LLM”) subsystem 402, a prediction subsystem 404, and/or a code level adjuster subsystem 406, among others.

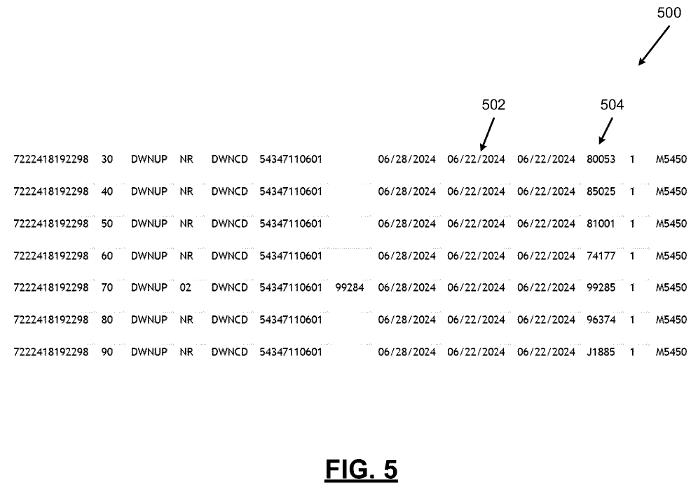

According to an example embodiment, the LLM subsystem 402 can comprise a generative artificial intelligence algorithm or any other artificial intelligence algorithm configured to ingest natural language. In some embodiments, the LLM subsystem 402 can receive a medical claim, such as a medical claim associated with a patient visiting an emergency room. An example medical claim 500 is illustrated in FIG. 5. The medical claim can include various information describing care provided to the patient. For example, the medical claim can include some or all of the following: the date and time when each medical procedure was performed 502, medical codes representing the medical procedures performed (e.g., CPT codes, revenue codes, diagnosis codes) 504, a diagnosis for the patient (which can be represented by a diagnosis code), demographics of the patient such as date of birth, gender, ethnicity, or any other status of the patient indicating a special need, a utilization level, and a written description of the care provided.

Referring again to FIG. 4, if the medical claim lacks procedure codes, the LLM subsystem 402 can add the codes to the medical claim as a separate column based on a written description of the care provided. Alternatively, the LLM subsystem 402 may convert codes from one coding standard to a different coding standard or add a second coding standard as another column to the medical claim.

The LLM subsystem 402 can implement various intelligence algorithms to improve and better learn from the received claims. In some embodiments, the LLM subsystem 402 can receive user feedback indicating that claims were incorrectly read or ingested by the LLM subsystem 402. In addition, the LLM system 402 may automatically receive the results of appeals conducted on appealed medical claims. If the appeal changed a result generated by the benefit manager system 102, then the LLM subsystem 402 may adjust feature vectors or other learning mechanics to enhance the LLM's intelligence.

The LLM subsystem 402 may be programmed or additionally learn that some combinations of claims are medically impossible or not recommended, thereby indicating a mistake or a need to adjust the level stored associated with a medical claim record. The LLM can read the diagnosis and the procedures stored in claim records and assign a weight for these types of records. The diagnosis and the procedures can be weighted separately. The LLM can develop logic rules to implement the adjustment of the level assigned to the claim. Claims are submitted by the provider device 134 with a level assigned to the medical record.

For example, the LLM system can account for known diagnosis and assign a weight. The known diagnosis can be known according to Centers for Medicare and Medicaid Services (CMS) or known diagnosis developed at the benefit manager device based on the medical records stored therein. In an example, the claim codes are related to chronic medical conditions or acute medical conditions. The claim codes are grouped by the benefit manager device and assigned a level code. This allows the benefit manager device to make a decision to upcode the level, leave the level as submitted by the provider, or downcode the level. Thus, a diagnosis code is mapped to a level, then the procedure code and diagnosis code can be used to adjust the level for a record under review.

For example, the LLM subsystem 402 may identify a medical code indicating a medical procedure performed on a child, but the date of birth identified by the claim suggests that the patient is an adult. Thus, the LLM subsystem 402 can change the code to the correct code for an adult and assume that the code for a child was entered by accident. As another example, the LLM subsystem 402 can review the dates for certain procedures and the number of similar procedures performed within a time frame. Some treatments or vaccinations can only be administered to a patient once per week or once per month or once per emergency room visit, but if the LLM subsystem 402 identifies that a one-a-week medical procedure was performed twice in the same day, then the second entry is likely either an error or fraud. Either way, the LLM subsystem 402 can disregard a second medical procedure or edit the claim so that the second medical procedure is not considered as part of the medical claim. In yet another embodiment, certain medical procedures, regardless of the number of times performed, may be counted only once. For example, eye irrigation or ear irrigation may incorrectly be counted twice because a patient has two ears or two eyes, but for the purposes of determining a care level, this procedure should only be counted once. The LLM subsystem 402 may be preprogrammed with some of this intelligence; it may learn certain rules by analyzing a large number of claims; or it may receive user feedback teaching additional rules later on.

The LLM subsystem 402 can also detect code changes to the medical procedure codes. According to an exemplary embodiment, the LLM subsystem 402 can compare a medical code representing a medical procedure in a medical claim to the long-form, written description of the medical procedure in the claim. The LLM subsystem 402 may find that the medical code and the long-form, written description of the procedure consistently do not match. In response, the LLM subsystem 402 can update its vector database because the LLM subsystem 402 determined that the medical code for that medical procedure changed. A similar process can occur anytime a new medical procedure, not previously stored as a vector in the vector database, is recognized in a claim.

The LLM subsystem 402 can include a diffuser or encoder that determines vectors in latent space. The LLM subsystem 402 can alter or change vector values in response to learning new information about medical claims. The training may occur from building sample sizes or from receiving user feedback. The LLM subsystem 402 can additionally add layers to a neural network or change connections between the nodes of the neural network in response to feedback or detected changes, which is described in more detail below. The LLM subsystem 402 continually trains and updates vectors in the LLM model to more accurately determine medical procedures in claims and levels of care determinations and special circumstances included in the medical claim. This allows the level of care data type can be corrected in the medical record.

The LLM subsystem 402 can also determine, based on written notes in the medical claim, special situations that cannot be captured by a set of medical codes. For example, whether a mandatory one-on-one nurse was required for a particular patient, whether security is necessary due to treatment of a dangerous individual, whether restraints on the patient were necessary during the medical procedure, whether the patient was suffering from a mental health episode, etc. The LLM subsystem 402 can review a medical claim and identify special situations that may impact scoring performed by the prediction subsystem 404.

The prediction subsystem 404 can receive the medical codes or a list of medical procedures identified by the LLM subsystem 402, and the prediction subsystem 404 can predict a level of care associated with the medical claim. For example, in an emergency room setting, hospitals may be reimbursed by insurance companies based on a level of care provided during an emergency room visit by a patient. As such, the level of care is an important determination in any medical claim. The level of care can apply to all emergency room situations or to only non-admitted patients who visit the emergency room. The level of care, also known as emergency room utilization level, may be included in a medical claim submitted to an insurance company. In other words, the hospital may self-assign a level of care and include that level of care with the medical claim. The prediction subsystem 404 can independently evaluate the level of care to determine if the hospital correctly self-evaluated the medical care provided, or whether the medical claim should be changed by either increasing or decreasing the level of care included in the medical claim.

The prediction subsystem 404 can determine a medical count indicating how much medical service was provided to a patient or how many medical procedures were performed. The count values can be based on medical codes (e.g., CPT codes), and each CPT code can have its own count value. In some embodiments, the prediction subsystem can apply weighting to each medical procedure performed. For example, diagnostic medical procedures may be weighted less heavily than therapeutic medical procedures, or vice versa. In other words, some medical procedures can have utilization points assigned that are weighted based on how time-intensive the medical procedure was, how expensive the medical procedure was, how many pieces of medical equipment were used to perform the procedure, how critical the medical procedure is, etc.

The prediction subsystem 404 can also apply across-the-board weighting to all the scores when a special situation arises. For example, if the patient is a child, the prediction subsystem 404 might increase the value (e.g. double) of all the medical procedure scores due to the special care necessary to treat a child. Across-the-board weighting increases can also apply to special needs individuals when the LLM subsystem 402 identifies a special needs situation in the claim. In some embodiments, the prediction subsystem applies as 2-8× weighting, depending on the circumstances and the patient. Patient demographics, such as age or gender, may also result in additional weighting to the count values. The additional weights come from clinical knowledge and may be preprogrammed into the prediction subsystem 404. Additionally, the prediction subsystem 404 may adjust weights as the prediction subsystem 404 learns from feedback received.

After assigning a score and weights to all the medical procedures included in the claim, the prediction subsystem 404 can determine the level of care recommended. In some embodiments, the prediction subsystem 404 can add all the weighted scores together to determine a summed, final score for all the medical procedures included in the medical claim. After determining the final score for the entire medical claim, the prediction subsystem 404 can compare the final score to ranges for each level of care. In some embodiments, a final score of 7 or less can result in a Level 3 level of care, a final score between 8 and 14 can result in a Level 4 level of care, and a final score over 14 can result in a Level 5 level of care. More or fewer levels of care may exist.

The prediction subsystem 404 can periodically review the scoring values assigned to each medical procedure in a database of medical procedures. For example, if a level of care determination made by the prediction subsystem 404 is overturned or changed during an appeal process, the prediction subsystem 404 can review the finding of the appeal and adjust weighting or scoring values so that the prediction subsystem 404 will not make the same mistake twice. In some embodiments, the LLM subsystem 402 can read the reasoning included in the appeal and provide suggestions to the prediction subsystem 404 for scores or weights that could be changed based on the reasoning included in the appeal. In this way, the prediction subsystem 404 responds to feedback and changes scoring or weighting or range values to account for incorrect predictions or changes to policy by the insurance company.

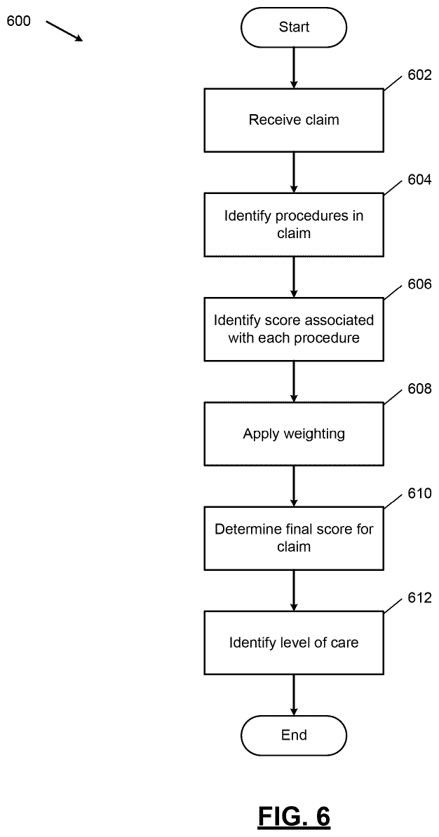

FIG. 6 illustrates an example method 600 illustrating the method for evaluating claims received by the benefit manager device 102. The method 600 can include the LLM subsystem 402 receiving a medical claim from a medical facility, such as a hospital submitting a medical claim for a non-admitted patient in step 602. The LLM subsystem 402 can read the claim and identify which medical procedures were listed in the medical claim by either referencing CPT codes or descriptions of the medical procedure performed in step 604, and the LLM subsystem 402 can provide the medical procedures in the claim to the prediction subsystem 404. The prediction subsystem 404 can assign a score to each procedure provided by the LLM subsystem 402 in step 606, the prediction subsystem 404 can apply weighting to the assigned scores in step 608, such as by multiplying the weighting value to some or all the assigned score values, and the prediction subsystem 404 can determine a final score for all the procedures in step 610, such as by adding all the weighted scores. Using the final score value, the prediction subsystem 404 can identify the level of care represented by the medical claim in step 612, such as by comparing the final score to ranges representing the various levels of care.

Referring again to FIG. 4, the code level adjuster subsystem 406 can receive a plurality of claims (e.g., claim 500) and automatically determine whether the emergency room code level (“ER”) code level assigned to each medical claim record requires adjustment (e.g., an increase or decrease in level). For example, the adjuster subsystem 406 can assess whether the ER code level for a given claim should be modified based at least in part on the diagnosis code(s) associated with the claim and may include other data related to the medical treatment and condition of the patient. In various implementations, the assigned ER code level data entry in the medical record reflects the determined level of care, which corresponds to the complexity and intensity of the patient encounter and is used to support further processing, e.g., adjudication, of the medical claim.

The code level adjuster subsystem 406 can also receive a plurality of claims (e.g., claim 500) and automatically determine whether the evaluation and management (“E/M”) code level assigned to each claim requires adjustment (e.g., an increase or decrease in level). For example, the adjuster subsystem 406 can assess whether the E/M code level for a given claim should be modified based at least in part on the diagnosis code(s) associated with the claim. In various implementations, the assigned E/M code level reflects the determined level of care, which corresponds to the complexity and intensity of the patient encounter and is used to support accurate further processing, e.g., adjudication, of the medical claim.

More specifically, E/M codes are CPT codes that classify patient encounters based on the type and complexity of care provided in various settings, such as office visits, hospital admissions, emergency services, and consultations. Each E/M code is associated with a specific level of service—such as low, moderate, or high complexity—determined by factors including medical decision-making, time spent, and the extent of history and examination performed. For example, office visit codes range across a spectrum of complexity, and hospital and emergency department codes follow similar tiered structures.

In various implementations, there are five levels of E/M codes. For example, Level 1 (e.g., code 99211) corresponds to minimal or no medical decision-making (“MDM”), typically involving brief visits that may not require a physician. Level 2 (e.g., code 99212) involves straightforward MDM. Level 3 (e.g., code 99213) involves low complexity MDM. Level 4 (e.g., codes 99214, 99204, 99244) involves moderate complexity MDM. Level 5 (e.g., codes 99215, 99205, 99245) involves high complexity MDM.

In various implementations, the code level adjuster subsystem 406, which operates automatically, provides significant advantages by improving accuracy and efficiency in medical coding. It can rapidly process thousands of claims, far exceeding the capacity of manual review, thereby ensuring consistent application of coding guidelines and reducing human errors. This scalability enables healthcare providers and payers to optimize reimbursement, enhance compliance, and minimize claim denials and audits.

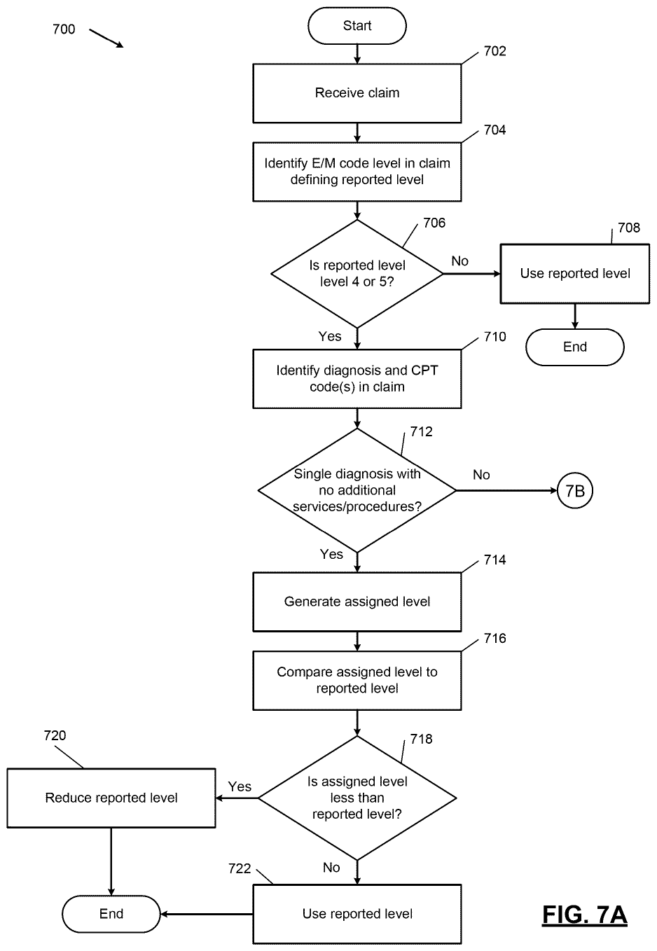

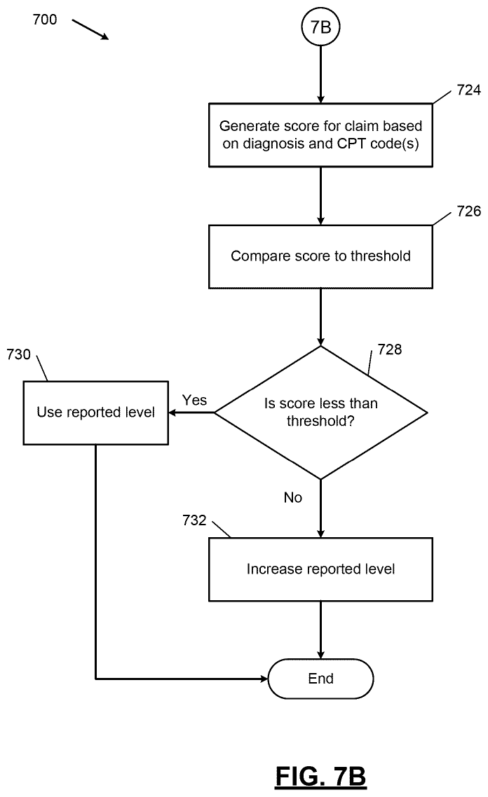

FIGS. 7A and 7B are flowcharts depicting an example method 700 for adjusting an E/M code level of a claim (e.g., claim 500). The method 700 may begin at 702. At 702, the code level adjuster subsystem 406 may receive a claim. The method 700 may proceed to 704. At 704, the adjuster subsystem 406 may identify the E/M code level in the claim. The identified E/M code level may define a reported level. The method 700 may proceed to 706.

At 706, the adjuster subsystem 406 may determine if the reported level is a level 4 (e.g., a moderate MDM level) or a level 5 (e.g., a high MDM level). If no at 706, the method 700 may procced to 708. At 708, the adjuster subsystem 406 may determine that the E/M code level does not require adjustment and then the method 700 may end. If yes at 706, the method 700 may proceed to 710.

At 710, the adjuster subsystem 406 may identify the diagnosis and the CPT codes in the claim. Then the method 710 may proceed to 712. At 712, the adjuster subsystem 406 may determine if the claim includes a single diagnosis with no additional services and/or procedures. In various implementations, the additional services/procedures refer to any extra medical treatments, diagnostic tests, or follow-up procedures provided beyond the primary reason for the visit. If yes at 712, the method 700 may proceed to 714. If no at 712, the method 700 may proceed to 724 of FIG. 7B.

At 714, the adjuster subsystem 406 may generate an assigned level (e.g., an E/M code level) based on the codes and information included in the claim. For example, the adjuster subsystem 406 may query the storage device 110 to retrieve a user-defined E/M code level associated with the diagnosis code in the claim, and use it to generate the assigned level. The method 700 may then proceed to 716.

At 716, the method 700 may compare the assigned level to the reported level and then proceed to 718. At 718, the adjuster subsystem 406 may determine whether the assigned level is less than the reported level. If yes, the method 700 may proceed to 720; otherwise, it may proceed to 722. At 720, the adjuster subsystem 406 may reduce the reported level. For example, in response to the assigned level being lower than the reported level, the adjuster subsystem 406 may decrease the E/M code level by one (e.g., from level 4 to level 3, or from level 5 to level 4). At 722, the adjuster subsystem 406 may determine that no adjustment is needed. For example, in response to the assigned level being equal to or greater than the reported level, the subsystem may retain the reported E/M code level, and the method 700 may end.

At 724, the adjuster subsystem 406 may generate a score for the claim based on the diagnosis and CPT codes included in the claim. For example, the adjuster subsystem 406 may determine a medical count indicating the extent of medical services provided to a patient or the number of medical procedures performed. The count values may be based on medical codes (e.g., CPT codes), with each CPT code assigned its own count value. In some embodiments, the adjuster subsystem 406 may apply weighting to each medical procedure performed (e.g., point of care (POC), blood/lab tests, immunizations/vaccines, radiology, procedures, therapy (excluding behavioral health), special care and testing, computer-aided testing, medications administered, durable medical equipment (DME), etc.). For example, each medical procedure may be weighted according to classifications such as minimal, low, moderate, or high, based on the nature of the service performed. In other words, some medical procedures may be assigned utilization points that are weighted according to factors such as the time intensity, cost, use of medical equipment, and criticality of the procedure. Then the method 700 may proceed to 726.

At 726, the adjuster subsystem 406 may compare the generated score to a user-defined threshold (e.g., 5 points, 10 points, etc.) The method 700 may then proceed to 728. At 728, the adjuster subsystem 406 may determine if the generated score is less than the threshold. If yes at 728, the method 700 may proceed to 730. If no at 728, the method 700 may proceed to 732.

At 730, the adjuster subsystem 406 may determine that the E/M code level does not require adjustment. For example, if the score is less than the threshold (e.g., 5 points), the adjuster subsystem 406 may retain the reported code level. The method 700 may then end.

At 732, the adjuster system 406 may increase the reported level. For example, in response to the score being greater than the threshold (e.g., 5 points, etc.), and the reported level being level 3, the adjuster subsystem 406 may increase the E/M code level to level 4. In another example, in response to the score being greater than the threshold (e.g., 10 points, etc.) and the reported level being level 4, the adjuster subsystem 406 may increase the E/M code level to a level 5. The method 700 may then end.

Examples of the present systems and methods address the highest two levels assigned to the medical records, e.g., levels 4 and 5. Levels 4 and 5 can be codes 99214, Established visit level 4 Moderate MDM; 99215, Established visit; level 5, High MDM; 99204, New visit; level 4 Moderate MDM; 99205, New visit level 5 Moderate MDM; 99244, New or established visit level 4 Moderate MDM; and 99245, New or established visit level 5 High MDM. These are the level that can be adjusted down in some examples.

In the examples where there is a single diagnose in the medical record, but has been reported in individual medical records for a same patient over a time period, e.g., one year, 18 months, or two years, this is a chronic condition that is assigned to the medical record. When the medical record has a level of 4 or 5, then the system assigns a level from the single diagnosis code list (which is soted in the storage device 110). If the assigned level is below the level that the provider assigned to the evaluation and evaluation and management code stored in the record, the system reduces the level one level. If the evaluation and management level assigned by the system is higher or the same level, the level in the record is not changed.

The scoring for step 724 can be created using an organized sortation of all the possible medical conditions into various classifications associated with at least levels 4 and 5. Certain acute conditions are coded down based on both the diagnosis and procedures. Certain chronic conditions are coded down based on both the diagnosis and procedure.

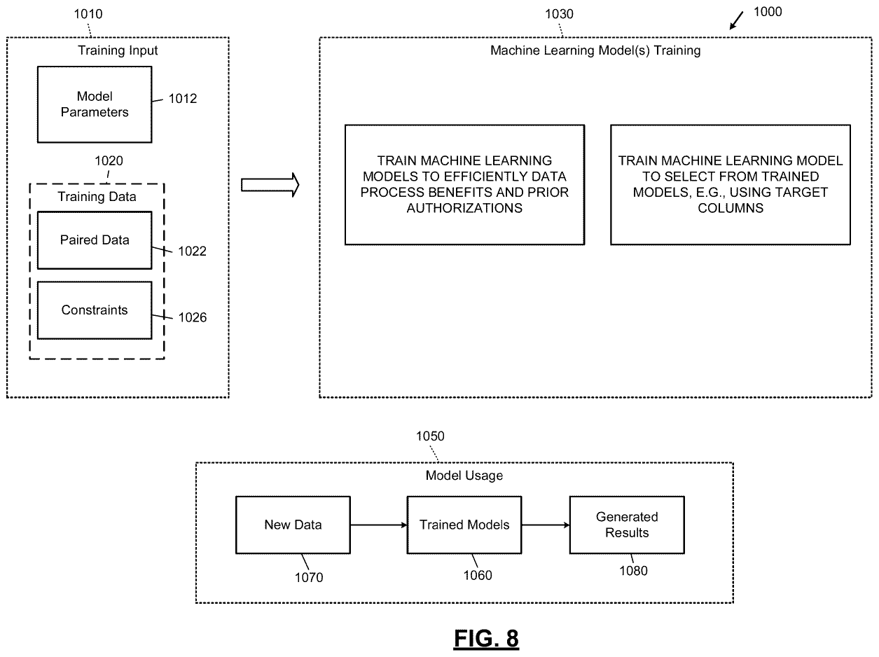

FIG. 8 is a block diagram of an example service of model platform 1000 that may be deployed within the system of FIG. 1, according to some examples. Training input 1010 includes model parameters 1012 and training data 1020 (e.g., data stored in storage 110) which may include paired training data sets 1022 (e.g., input-output training pairs) and constraints 1026. The model platform 1000 can be part of the benefit manager device, which can provide claim evaluation functionality. Model parameters 1012 stores or provides the parameters or coefficients of corresponding ones of machine learning models. During training, these parameters 1012 are adapted based on the input-output training pairs of the training data sets 1022. After the parameters 1012 are adapted (after training), the parameters are used by trained models 1060 to implement the trained machine learning models on a new set of data 1070.

Training data 1020 includes constraints 1026, which may define the constraints of a given patient information feature or a given benefit plan features. The paired training data sets 1022 may include sets of input-output pairs, such as pairs of a plurality of patient information features and features of inquiries associated with the patient information. Some components of training input 1010 may be stored separately at a different off-site facility or facility than other components.

Machine learning model(s) training 1030 trains one or more machine learning techniques based on the sets of input-output pairs of paired training data sets 1022. For example, model training 1030 may train the machine learning (ML) model parameters 1012 by minimizing a loss function based on one or more ground-truth data.

The ML models can include any one or combination of classifiers, LLMs, or neural networks, such as an artificial neural network, a convolutional neural network, an adversarial network, a generative adversarial network, a deep feed-forward network, a radial basis network, a recurrent neural network, a long/short term memory network, a gated recurrent unit, an autoencoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, a first ML model of the ML models can be applied to a training batch of patient information features to estimate or generate a prediction of inquiries associated with the claim evaluation and level of care determination. In some implementations, a derivative of a loss function is computed based on a comparison of the estimated prediction of the level of care inquiries and the ground truth resulting from those inquiries, and parameters of the first ML model are updated based on the computed derivative of the loss function. The result of minimizing the loss function for multiple sets of training data trains adapts, or optimizes the model parameters 1012 of the corresponding first ML model. In this way, the first ML model is trained to establish a relationship between a plurality of training information and ground-truth results. This system can be repeated for each type of claim evaluation that may be requested. The claim evaluation available may change for each individual, each plan covering the individual, data from the provider requesting the claim evaluation, and the medical condition experienced by the individual.

A second ML model of the ML models can be trained to select the correct ML model to be used in the claim evaluation and level of care determination. The second ML model can access data on which the first models work, the success of the model in correctly conduct a claim evaluation and the level of care determination. The second ML model can be used to generate a selection system or AI bot to select the correct model. Thus, the present system can implement and select between multiple machine models generated by ML, agentic actors, or LLMs to provide different types of records processing to improve efficiencies within the computing systems, e.g., by reducing the number of tokens required to run certain data through the computing engine for processing the machine-readable medical records. This in turn will reduce processing time and the energy consumed to process the medical records as described herein.

After the machine learning models are trained, new data 1070, including all the features related to level of care determination, are received and/or derived by the claim evaluation platform 1000. The first trained machine learning model may be applied to the new data 1070 to generate results 1080, including a prediction of level of care for a medical claim. The prompts are applied to the second trained machine learning model to perform tasks for evaluation and selection of models.

The multiple machine model embodiment can align the medical records being processed with the model that has been trained and post trained by generative artificial intelligence (a large language model (LLM) or a large multimodal model (LMM)) with the principles of a specific domain, e.g., correction of the medical record by automatically adjusting its coding level. The system and method may also generate aligning processes that may be used to post-train an already trained generative artificial intelligence system or fine tune the training of the generative artificial intelligence system to correct machine models that more closely align that generative artificial intelligence system with the principles of the specific domain.

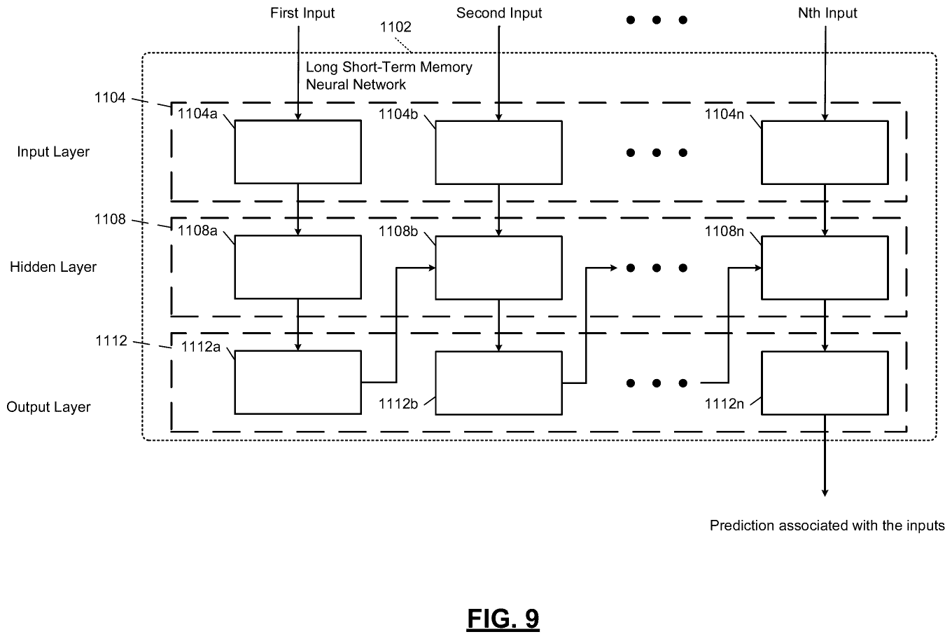

FIG. 9 is a functional block diagram of an example neural network 1102 that can be used for the inference engine or other functions (e.g., engines) as described herein to produce a predictive model. The predictive model can identify or generate inquiries associated with patient information. In an example, the neural network 1102 can be a LSTM neural network. In an example, the neural network 1102 can be a recurrent neural network (RNN). The example neural network 1102 may be used to implement the machine learning as described herein, and various implementations may use other types of machine learning networks. The neural network 1102 includes an input layer 1104, a hidden layer 1108, and an output layer 1112. The input layer 1104 includes inputs 1104a, 1104b . . . 1104n. The hidden layer 1108 includes neurons 1108a, 1108b . . . 1108n. The output layer 1112 includes outputs 1112a, 1112b . . . 1112n.

Each neuron of the hidden layer 1108 receives an input from the input layer 1104 and outputs a value to the corresponding output in the output layer 1112. For example, the neuron 1108a receives an input from the input 1104a and outputs a value to the output 1112a. Each neuron, other than the neuron 1108a, also receives an output of a previous neuron as an input. For example, the neuron 1108b receives inputs from the input 1104b and the output 1112a. In this way the output of each neuron is fed forward to the next neuron in the hidden layer 1108. The last output 1112n in the output layer 1112 outputs a probability associated with the inputs 1104a-1104n. Although the input layer 1104, the hidden layer 1108, and the output layer 1112 are depicted as each including three elements, each layer may contain any number of elements. Neurons can include one or more adjustable parameters, weights, rules, criteria, or the like.

In various implementations, each layer of the neural network 1102 must include the same number of elements as each of the other layers of the neural network 1102. For example, training features (e.g., collection of patient information associated with a first set of ground truth inquiries) may be processed to create the inputs 1104a-1104n.

The neural network 1102 may implement a first model to produce a set of inquiries. More specifically, the inputs 1104a-1104n can include fields of the patient information as data features (binary, vectors, factors or the like) stored in the storage device 110. The features of the medical claim can be provided to neurons 1108a-1108n for analysis and connections between the target columns, the data on which the level of care determination is based, which model should be used for a particular medical condition, or the performance of the models individually. The neurons 1108a-1108n, upon finding connections, provides the potential connections as outputs to the output layer 1112, which determines a set of inquiries associated with the claim information.

The neural network 1102 can perform any of the above calculations. The output of the neural network 1102 can be used to control an LLM to retrieve the appropriate set of information. In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 1104a is connected to each of neurons 1108a, 1108b . . . 1108n.

The present disclosure includes embodiments that identify claim records for downcoding or upcoding the level associated with the record. The machine decision to change the level can be based on the diagnosis, the procedure(s), or both in the claim record. Both of these inputs can be assigned a weight. If the system does not have adequate data in the record, it can request documentation from the provider in order to support the claim as it was originally sent to benefit manager. For every encounter a patient has with a physician in an office, clinic, hospital, or emergency room, there is a specific evaluation and management current procedural terminology code that represents the amount of time spent with the patient during the encounter, whether the patient is a new or established patient, the severity of their illness or injury and the complexity of the encounter (e.g., multiple conditions or chronic diseases). These codes are ranked from level 1 to level 5 with 5 being the highest complexity and time spent with the patient. When a provider submits a claim for a level 4 or 5 evaluation and management code, that does not have the proper diagnosis included in the claim record, the benefit provider can downcode the claim record by one level which results in a reduction of payment to the provider. The present systems and methods can correct a level of a medical record to ensure accurate records in the database.

In an example, a provider device 134 submits a 99214 code for an office visit where the patient was diagnosed with the flu. Since 99214 is a level 4 evaluation and management CPT code, the benefit provider reduces the evaluation and management code to a level 3 equivalent and then further adjudicates the adjusted medical records. Rather than pending the claim for request for additional information from the provider, the benefit manager device can process the claim record and pay the claim at the lower evaluation and management CPT code contracted rate and if the provider disagrees, the provider device 134 can submit documentation to prove the claim record should be adjudicated as it was originally submitted.

If a claim is received that contains an evaluation and management code that does not align with the diagnosis billed, the benefit manager device will downcode the claim record and notify the provider device 134 via remark codes. The responsibility is then on the provider device 134 to review the remittance advice/explanation of payment to decide whether the provider agrees or disagrees with the adjustment on the claim record.

In view of the following, the systems and methods described herein can implement artificial intelligence (e.g., and LLM) to review medical claims submitted to an insurance provider. In terms of volume, the number of medical claims submitted to the insurance company can be in the tens of thousands per day or even hour, depending on the number of insured members. The LLM can read all information in the medical claim, including the medical procedures performed, the codes associated with those procedures, and a self-assigned E/M code level. Because the systems and methods implement an LLM or other artificial intelligence, the systems and methods can fully review the medical claim and perform a calculation to determine if the E/M code level was properly or improperly self-assigned and further increase or decrease the E/M code level when the self-assigned E/M code level was improperly assigned.

Moreover, because the systems and methods implement an LLM or other artificial intelligence, the systems and methods can ingest natural language to ensure that the medical code assigned to a medical procedure matches the natural language description of the medical procedure.

The LLM can further train on appeal results to better improve the accuracy and functioning of the LLM. The appeal results are based on previously processed claim records, which can have data types the same as the current medical records being evaluated and possibly adjusted as needed. The appeal results can be used to alter the weighting at a neuron in the layers of the LLM or artificial neural network.

The computing engine can include a large language model (LLM) subsystem, a prediction subsystem, and a level adjuster subsystem in communication with each other. The large language model (LLM) subsystem can be configured to (a) adapt code or instructions to learn from a training dataset to generate a trained LLM, (b) receive a medical claim comprising procedure data indicating medical procedures performed on an individual during a medical encounter, (c) ingest the data indicating the medical procedures to generate a list of medical procedures identified in the medical claim and a self-assigned level of care, and (d) alter feature vectors in response to new data or additional training. The prediction subsystem can be configured to (a) determine a respective medical score for each procedure in the list of medical procedures generated by the trained LLM, (b) apply a respective weight to each respective medical score, (c) determine a final score for the medical encounter based on the respective weight applied to each respective medical score, (d) compare the final score to one or more thresholds to determine a predicted level of care, and (e) compare the predicted level of care to the self-assigned level of care. The level adjuster subsystem can be configured to amend the medical claim with the predicted level of care when the predicted level of care is different than the self-assigned level of care. The adjustment of the level can be done automatically within the computing engine. The weighting at any individual neuron in the LLM, ANN, or model can also be automatically updated within the computing engine.

Furthermore, the systems and methods described herein can process a large volume of medical claims (hundreds, thousands, tens of thousands, hundreds of thousands) on a daily basis and still look for inconsistencies or incorrect leveling assigned by a provider who submitted the medical claim. Moreover, the systems and methods improve the functioning of the computer systems described herein by implementing continuous training to the LLM subsystem 402, resulting in improved accuracy and faster determinations, and also continuous updates to the prediction subsystem 404 by adjusting weighting and valuation that can result in more accurate predictions, thereby leading to fewer appeal processes and additional reprocessing due to errors.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “coupled,” “engaged,” “adjacent,” “next to,” “on top of,” “above,” “below,” and “disposed.” Unless explicitly described as being “direct,” when a relationship between first and second elements is described in the above disclosure, that relationship encompasses a direct relationship where no other intervening elements are present between the first and second elements as well as an indirect relationship where one or more intervening elements are present (either spatially or functionally) between the first and second elements.

The term “set” generally means a grouping of one or more elements. The elements of a set do not necessarily need to have any characteristics in common or otherwise belong together. However, in various implementations a “set” may, in certain circumstances, be the empty set (in other words, the set has zero elements in those circumstances). As an example, a set of search results resulting from a query may, depending on the query, be the empty set. In contexts where it is not otherwise clear, the term “non-empty set” can be used to explicitly denote exclusion of the empty set —that is, a non-empty set will always have one or more elements.

A “subset” of a first set generally includes some of the elements of the first set. In various implementations, a subset of the first set is not necessarily a proper subset: in certain circumstances, the subset may be coextensive with (equal to) the first set (in other words, the subset may include the same elements as the first set). In contexts where it is not otherwise clear, the term “proper subset” can be used to explicitly denote that a subset of the first set must exclude at least one of the elements of the first set. Further, in various implementations, the term “subset” does not necessarily exclude the empty set. As an example, consider a set of candidates that was selected based on first criteria and a subset of the set of candidates that was selected based on second criteria; if no elements of the set of candidates met the second criteria, the subset may be the empty set. In contexts where it is not otherwise clear, the term “non-empty subset” can be used to explicitly denote exclusion of the empty set.

The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgments of, the information to element A.

In this application, including the definitions below, the term “module” can be replaced with the term “controller” or the term “circuit.” In this application, the term “controller” can be replaced with the term “module.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); processor hardware (shared, dedicated, or group) that executes code; memory hardware (shared, dedicated, or group) that is coupled with the processor hardware and stores code executed by the processor hardware; other suitable hardware components that provide the described functionality; or a combination of some or all of the above, such as in a system-on-chip. These structures includes hardware circuitry that implement the computing engine for data processing as described herein.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2020 (also known as the WIFI wireless networking standard) and IEEE Standard 802.3-2018 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

Some or all hardware features of a module may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a module may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The memory hardware may also store data together with or separate from the code. Shared memory hardware encompasses a single memory device that stores some or all code from multiple modules. One example of shared memory hardware may be level 1 cache on or near a microprocessor die, which may store code from multiple modules. Another example of shared memory hardware may be persistent storage, such as a solid state drive (SSD) or magnetic hard disk drive (HDD), which may store code from multiple modules. Group memory hardware encompasses a memory device that, in combination with other memory devices, stores some or all code from one or more modules. One example of group memory hardware is a storage area network (SAN), which may store code of a particular module across multiple physical devices. Another example of group memory hardware is random access memory of each of a set of servers that, in combination, store code of a particular module. The term memory hardware is a subset of the term computer-readable medium.

The apparatuses and methods described in this application may be partially or fully implemented by a special-purpose computer created by configuring a general-purpose computer to execute one or more particular functions embodied in computer programs. Such apparatuses and methods may be described as computerized or computer-implemented apparatuses and methods, e.g., a computing engine, artificial neural network circuitry, or the like. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special-purpose computer, device drivers that interact with particular devices of the special-purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

The term non-transitory computer-readable medium does not encompass transitory electrical or electromagnetic signals propagating through a medium (such as on a carrier wave). Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc). Another example of non-transitory computer-readable medium may include machine readable structures that can be read by a computing engine or communication machine.

1. A system comprising:
a large language model (LLM) subsystem configured to (a) adapt code or instructions to learn from a training dataset to generate a trained LLM, (b) receive a medical claim comprising procedure data indicating medical procedures performed on an individual during a medical encounter, (c) ingest the data indicating the medical procedures to generate a list of medical procedures identified in the medical claim and a self-assigned level of care, and (d) alter feature vectors in response to new data or additional training;
a prediction subsystem configured to (a) determine a respective medical score for each procedure in the list of medical procedures generated by the trained LLM, (b) apply a respective weight to each respective medical score, (c) determine a final score for the medical encounter based on the respective weight applied to each respective medical score, (d) compare the final score to one or more thresholds to determine a predicted level of care, and (e) compare the predicted level of care to the self-assigned level of care; and
a level adjuster subsystem configured to amend the medical claim with the predicted level of care when the predicted level of care is different than the self-assigned level of care.

2. The system of claim 1 wherein the procedure data indicating medical procedures comprises procedure codes.

3. The system of claim 1, wherein the procedure data indicating medical procedures comprises a natural language written description of the care provided, and wherein the LLM subsystem is further configured to determine a procedure code from the natural language ingested written description of the care provided.

4. The system of claim 3, wherein the procedure data indicating medical procedures comprises procedure codes and a natural language written description of the care provided, and wherein the LLM subsystem is further configured to compare the natural language written description of the care provided to a description of the procedure code to ensure that the procedure code matches the natural language written description of the care provided.

5. The system of claim 1, wherein the LLM subsystem is further configured to ingest data indicating at least one diagnosis, and wherein the prediction subsystem apply the respective weight based on the data indicating at least one diagnosis.

6. The system of claim 1, wherein the LLM subsystem is further configured to ingest data indicating a special condition of the individual, and wherein the prediction subsystem is further configured to apply the respective weight based on the special condition.

7. The system of claim 6, wherein the special condition is an age, gender, or medical condition of the individual.

8. The system of claim 5, wherein the level adjuster subsystem is further configured to determine whether the data indicating at least one diagnosis indicates a single diagnosis or multiple diagnoses.

9. The system of claim 8, wherein, when the data indicating at least one diagnosis indicates multiple diagnoses, the level adjuster subsystem is further configured to determine the predicted level of care based on the list of medical procedures generated by the trained LLM and the respective weight.

10. The system of claim 8, wherein, when the data indicating at least one diagnosis indicates the single diagnosis, the level adjuster subsystem is further configured to determine the predicted level of care based on the single diagnosis.

11. A method of using a large language model (LLM) to process data comprising:
the LLM adapting code or instructions to learn from a training dataset to generate a trained LLM;
the trained LLM receiving a medical claim comprising procedure data indicating medical procedures performed on an individual during a medical encounter and a self-assigned level of care;
the trained LLM ingesting the data indicating the medical procedures to generate a list of medical procedures identified in the medical claim;
the trained LLM altering feature vectors in response to new data or additional training;
a prediction subsystem determining a respective medical score for each procedure in the list of medical procedures generated by the trained LLM;
the prediction subsystem applying a respective weight to each respective medical score;
the prediction subsystem determining a final score for the medical encounter based on the respective weight applied to each respective medical score;
the prediction subsystem comparing the final score to one or more thresholds to determine a predicted level of care, and
the prediction subsystem comparing the predicted level of care to the self-assigned level of care; and
a level adjuster subsystem amending the medical claim with the predicted level of care when the predicted level of care is different than the self-assigned level of care.

12. The method of claim 11, wherein the procedure data indicating medical procedures comprises procedure codes.

13. The method of claim 11, wherein the procedure data indicating medical procedures comprises a natural language written description of the care provided; and further comprising:
the LLM subsystem determining a procedure code from the natural language ingested written description of the care provided.

14. The method of claim 13, wherein the procedure data indicating medical procedures comprises procedure codes and a natural language written description of the care provided; and further comprising:
the LLM subsystem comparing the natural language written description of the care provided to a description of the procedure code to ensure that the procedure code matches the natural language written description of the care provided.

15. The method of claim 11, further comprising:
LLM subsystem ingesting data indicating at least one diagnosis; and
the prediction subsystem applying the respective weight based on the data indicating at least one diagnosis.

16. The method of claim 11, further comprising:
the LLM subsystem ingesting data indicating a special condition of the individual; and
the prediction subsystem applying the respective weight based on the special condition.

17. The method of claim 16, wherein the special condition is an age, gender, or medical condition of the individual.

18. The method of claim 15, further comprising:
the level adjuster subsystem determining whether the data indicating at least one diagnosis indicates a single diagnosis or multiple diagnoses.

19. The method of claim 18, wherein, when the data indicating at least one diagnosis indicates multiple diagnoses, the level adjuster subsystem is further configured to determine the predicted level of care based on the list of medical procedures generated by the trained LLM and the respective weight.

20. The method of claim 18, wherein, when the data indicating at least one diagnosis indicates the single diagnosis, the level adjuster subsystem is further configured to determine the predicted level of care based on the single diagnosis.

21. A method of processing data comprising:
an artificial intelligence (AI) subsystem adapting code or instructions to learn from a training dataset to generate a trained LLM;
the AI subsystem receiving a medical claim comprising procedure data indicating medical procedures performed on an individual during a medical encounter and a self-assigned level of care;
the AI subsystem ingesting the data indicating the medical procedures to generate a list of medical procedures identified in the medical claim;
the AI subsystem altering feature vectors in response to new data or additional training;
a prediction subsystem determining a respective medical score for each procedure in the list of medical procedures generated by the AI subsystem;
the prediction subsystem applying a respective weight to each respective medical score;
the prediction subsystem determining a final score for the medical encounter based on the respective weight applied to each respective medical score;
the prediction subsystem comparing the final score to one or more thresholds to determine a predicted level of care, and
the prediction subsystem comparing the predicted level of care to the self-assigned level of care; and
a level adjuster subsystem amending the medical claim with the predicted level of care when the predicted level of care is different than the self-assigned level of care.
